(19) United States
(12) Patent Application Publication
(10) Pub. No.: US 2011/0301153 A1
(43) Pub. Date: Dec. 8, 2011

(54) METHODS OF TREATING CANCER

(75) Inventors: Craig B. Thompson, Merion Station, PA (US); David R. Wise, Philadelphia, PA (US)

(73) Assignee: The Trustees of the University of Pennsylvania, Philadelphia, PA (US)

(21) Appl. No.: 13/129,679

(22) PCT Filed: Nov. 16, 2009
(86) PCT No.: PCT/US09/64569

§ 371 Date: Aug. 10, 2011

Related U.S. Application Data
(60) Provisional application No. 61/115,273, filed on Nov. 17, 2008.

(51) Int. Cl. A61K 31/5415; A61P 35/00; C12N 5/09; A61K 31/195; A61K 31/192

(52) U.S. Cl. 514/224.8; 514/561; 514/570; 435/375

(57) ABSTRACT

The invention concerns methods of treating cancer comprising contacting said tumors with a chemical inhibitor of glutaminolysis. These inhibitors include amino-oxyacetate, phenylbutyrate, phenylacetate, and 3,7-bis(dimethylamino)-phenazathionium chloride (methylene blue).

### RELATED APPLICATIONS

This application claims benefit of U.S. Patent Application No. 61/115,273, filed Nov. 17, 2008, the disclosure of which is incorporated by reference herein in its entirety.

### STATEMENT OF GOVERNMENT SUPPORT

This invention was made using government support from NIH Grant K08 DK072565, HD26979 and NS054900. Accordingly, the United States Government may have certain rights in the invention described herein.

### TECHNICAL FIELD

The present invention concerns methods of treating cancer that disrupt glutamine uptake and metabolism of cancer cells.

### BACKGROUND

Mammalian cells fuel their growth and proliferation through the catabolism of two main substrates: glucose and glutamine. Most of the remaining metabolites taken up by proliferating cells are not catabolized, but instead are utilized as building blocks during anabolic macromolecular synthesis.

Many cancer cell lines depend on a high rate of glucose uptake and metabolism to maintain their viability despite being maintained in an oxygen-replete environment. See, Warburg O (1956) Science 123(3191):309-314. This metabolic phenotype, first observed by Otto Warburg, has been termed aerobic glycolysis. Warburg O (1925) Journal of Molecular Medicine 4(12):534. Initially, this high rate of glycolysis was believed to result from mutations that impair the ability of cancer cells to carry out oxidative phosphorylation. Warburg O (1956) Science 124(3215):269-270. However, such defects appear to be rare in spontaneously arising tumors. Kroemer G & Pouyssegur J (2008) Cancer Cell 13(6):472. Recent studies have suggested that activating mutations in phosphoinositol 3-kinase (PI3K) and its downstream effector AKT induce the transformed cell to take up glucose in excess of its bioenergetic needs. Elstrom R L, et al. (2004) Cancer Res 64(11):3892-3899. The resulting high rate of glycolytic metabolism leads to the conversion of mitochondria into synthetic organelles that support glucose-dependent lipid synthesis and non-essential amino acid production. Lum J J, et al. (2007) Genes Dev 21(9):1037-1049 and Bauer D E, et al. (2005) Oncogene 24(41):6314. Glycolytic pyruvate that accumulates in excess of a cell's bioenergetic and synthetic needs is converted to lactate and secreted. A consequence of this metabolic conversion is that cells become addicted to glucose for their ATP production and survival as available lipids and amino acids are redirected from use as bioenergetic substrates and committed to use in anabolic synthesis. Elstrom R L, et al. (2004) Cancer Res 64(11):3892-3899. These data suggest that cancer cell nutrient uptake and metabolism may be under the direct control of the oncogenic signaling pathways that transform the cell. Strategies to exploit the glucose addiction of cells transformed by PI3K mutation for cancer therapy are currently being investigated. Kroemer G & Pouyssegur J (2008) Cancer Cell 13(6):472.

In addition to glucose, glutamine can be an essential nutrient for cell growth and viability. Coles N W & Johnstone R M (1962) Biochem J 83:284-291 and Eagle H (1955) Science 122(3168):501-514. In vitro addiction to glutamine as a bioenergetic substrate was first observed in HeLa cells but it was not found to be a universal property of cancer cell lines. In cancer patients, some tumors have been reported to consume such an abundance of glutamine that they depress plasma glutamine levels. Suzannec Klimberg V & McClellan J L (1996) The American Journal of Surgery 172(5):418 and Chen M K, et al., (1993) Ann Surg 217(6):655-666; discussion 666-657. Despite these observations, the high rate of glutamine metabolism and addiction exhibited by some cancer cells is poorly understood. Recently, we reported that glioma cells can exhibit glutamine uptake and metabolism that exceeds the cell's use of glutamine for protein and nucleotide biosynthesis. DeBerardinis R J, et al. (2007) Proceedings of the National Academy of Sciences 104(49):19345-19350. In such cells, the excess glutamine metabolites produced were found to be secreted as either lactate or alanine. This high rate of glutaminolysis was found to be beneficial because it provided the cell a high rate of NADPH production that was utilized to fuel lipid and nucleotide biosynthesis (see FIG. 7 for schematic of glutaminolytic pathway). However, not all tumor cells exhibit glutaminolysis. This suggested that the use of glutamine as a bioenergetic substrate is not induced as an indirect consequence of cell growth, but as a direct consequence of a specific oncogenic event.

Despite the progress that has been made in the study of the proliferation of cancer cells, there is a need for treatments that prevent or treat the disease.

### SUMMARY

In some aspects, the invention concerns methods of treating cancer comprising contacting the cancer cells with a chemical inhibitor of glutaminolysis. Chemical inhibitors of glutaminolysis include amino-oxyacetate, phenylbutyrate, phenylacetate, and 3,7-bis(dimethylamino)-phenazathionium chloride (methylene blue).

In some embodiments, the treated cancer is a solid cancer such as cancer of the lung, breast, colon, or prostate. In other aspects, the cancer is lymphoma, bronchoalvedar cell lung carcinoma, or carcinold tumor.

The invention also concerns methods of treating a Myc-induced tumor comprising contacting the tumors with a chemical inhibitor of glutaminolysis. Other aspects include methods of inhibiting glutaminolysis in a Myc expressing cell comprising contacting cancer cells with one or more of amino-oxyacetate, phenylbutyrate, phenylacetate, and 3,7-bis(dimethylamino)-phenazathionium chloride (methylene blue). Yet another aspect includes methods of preventing cancer comprising contacting Myc expressing cells with one or more of amino-oxyacetate, phenylbutyrate, phenylacetate, and 3,7-bis(dimethylamino)-phenazathionium chloride (methylene blue).

In certain aspects, the present invention disclosure relates to the use of pharmacological agents, i.e., amino-oxyacetate (AOA), phenylbutyrate/phenylacetate, and methylene blue, for the treatment of tumors with accelerated rates of glutamine uptake and catabolism, especially those driven by the oncogene Myc. These agents interrupt the high rate of glutamine-dependent metabolism critical for the survival of Myc-overexpressing tumors. This specificity should maximize their anti-tumor effect while minimizing side effects. It has long been suspected that human tumors and tumor cells rely on aspects of glutamine metabolism to support their viability and growth. We have discovered that Myc enables cancer cells to import and catabolize glutamine to produce ATP and NADPH to fuel cancer cell growth and preserve viability. The high rate of glutamine metabolism is therefore diagnostic for the activation of the Myc family of oncogenes and we have found that the impairment of glutamine-dependent metabolism is deleterious to the growth of Myc-transformed cells. The compounds detailed herein have already been approved for other clinical applications and would work through the depletion of plasma glutamine through phenylbutyrate/phenylacetate, inhibition of glutamine catabolism (AOA), or depletion of cellular NADPH (methylene blue).

### BRIEF DESCRIPTION OF THE DRAWINGS

FIG. 1 concerns glutamine catabolism in the human glioma line SF188. (A) Protein synthesis is a minor fate of glutamine carbon. SF188 cells were cultured in medium supplemented with 0.01% [¹⁴C-U₅]glutamine relative to unenriched glutamine for 4 h. [¹⁴C-U₅]glutamine in SSA precipitated protein (striped bar) and total glutamine consumed from the medium (grey bar) are presented as the mean±standard deviation (SD) of four independent experiments. (B) The requirement for glutamine can be satisfied by alpha-ketoglutarate. SF188 cells were allowed to plate in complete medium and then cultured in either glutamine-depleted medium (−glutamine), complete medium (+glutamine), or glutamine-depleted medium supplemented with 7 mM dimethyl α-ketoglutarate (−glutamine +α-ketoglutarate). Cell viability was determined at the time points shown by trypan blue dye exclusion. The data presented are the mean±SD of triplicate samples. Representative data from one of three independent experiments are shown.

FIG. 2 shows PI3K/Akt signaling regulates the consumption of glucose but not of glutamine. SF188 cells stably expressing Bcl-x_{L} were treated with AktiVIII at doses ranging from 0-20 μM. Medium was collected and analyzed for glucose, lactate, glutamine, and ammonia. The rates shown were calculated from the difference in metabolite concentration between the medium at the time point shown and fresh medium. The data points presented are the mean±SD of triplicate samples.

FIG. 3 shows Myc activates the transcription of genes involved in glutamine uptake and metabolism. (A) Myc protein is overexpressed in SF188 cells. Western blot reveals overexpression of c-Myc in SF188 glioblastoma cells compared to MEF and another glioblastoma cell line, LN229. (B) Myc is required for glutamine metabolism. SF188 cells were transduced with either lentiviral shRNA against Myc (shMYC) or Luciferase (shCTRL) for 3 days. Glutamine and ammonium levels in the medium were analyzed using the Nova Flex and are presented as the mean±SD of triplicate samples. Data from one of five independent experiments are shown. Knockdown of Myc protein is depicted in (A). (C) Myc is required for the expression of the proximal enzymes of glutaminolysis. RNA was extracted from shMYC and shCTRL cells and quantified using quantitative RT-PCR (qPCR). The bars shown are normalized to a β-actin control and represent the mean±SD of triplicate samples. Representative data from one of two independent experiments are shown. EIF1A is included as a negative control. (D) Myc is enriched at the regulatory binding sites of genes involved in glutamine uptake. Sheared chromatin from fixed and lysed SF188 cells was immuno-precipitated using the antibodies indicated. Precipitated DNA fragments were quantified by qPCR. The data presented are the mean±SD of triplicate samples. The schematic shows the location of Myc-bound E-box elements within the genomic loci of ASCT2 and SN2.

FIG. 4 shows that Myc activates glutaminolysis in MEF. (A) Oncogenic levels of Myc induce the expression of genes involved in glutaminolysis. qPCR analysis of target genes from total RNA isolated from MEF MycER treated with 200 nM 4-hydroxytamoxifen (4-OHT) or vehicle (EtOH) for 24 h. The bars shown are normalized to an internal β-actin control and represent the mean±SD of triplicate samples. Representative data from one of three independent experiments are shown. (B) Oncogenic levels of Myc induce glutamine uptake. MEF MycER treated as in (A) were cultured for 1 min with medium supplemented with [U-¹⁴C₅]glutamine. Uptake of the label was quantified by scintillation counting of the cellular lysate. The data presented are the mean±SD of triplicate samples. (C) Oncogenic levels of Myc induce flux through glutaminase. MEF MycER treated as in (A) were cultured for 8 h with medium supplemented with L-[γ-¹⁵N]glutamine. Glutaminase activity was determined by measuring the isotopic enrichment of ¹⁵N in NH₄⁺ in the culture medium by GC-MS. The bars shown represent the mean±standard deviation (SD) of triplicate cultures. (D) Oncogenic levels of Myc induce the flux of glutamine into lactate. MEF MycER treated as in (A) were cultured for 6 h in medium supplemented with 4 mM [U-¹³C₅]glutamine. The medium was subsequently removed and analyzed with ¹³C NMR spectroscopy. [2,3-¹³C]lactate is metabolically derived from [U-¹³C₅]glutamine, while [3-¹³C]lactate is metabolically derived from the natural abundance of [1-¹³C]glucose and [6-¹³C]glucose. The data presented are the mean±SD of triplicate samples. (E) Oncogenic levels of Myc induce the consumption of glutamine from the medium. The glutamine concentration in medium from MEF MycER treated as in (A) was analyzed at the time points shown by the Nova Flex. The data points shown represent the mean±SD of triplicate samples.

FIG. 5 shows Myc diverts glucose away from mitochondrial metabolism in MEF. (A) Oncogenic levels of Myc suppress the contribution of glucose to phospholipid synthesis. MEF MycER treated as in FIG. 4A were cultured with medium supplemented with D-[U-¹⁴C]-glucose for 8 h. After the culture period, lipids were harvested and ¹⁴C enrichment in phospholipids (PL) was determined by scintillation counting. The bars shown represent the mean±SD of triplicate samples. Representative data from one of three experiments are shown. (B) Oncogenic levels of Myc induce lactate production. The lactate concentration in the medium from MEF MycER treated with 4-OHT or EtOH for 18 h was quantified using the Nova Flex Metabolite Analyzer. Each time point is the mean±SD of triplicate samples. Representative data from one of three experiments are shown. (C) Glutamine's contribution to phospholipid synthesis is maintained in the presence of oncogenic levels of Myc. MEF MycER treated as in FIG. 5A were cultured with medium supplemented with L-[U-¹⁴C]-glutamine for 8 h. After the culture period, lipids were harvested and ¹⁴C enrichment in PL was determined by scintillation counting. The bars shown represent the mean±SD of triplicate samples. Representative data from one of three independent experiments are shown.

FIG. 6 shows that the glutamine addiction exhibited by SF188 glioma cells is Myc-dependent. (A) Myc-suppressed SF188 cells are resistant to glutamine starvation. shMYC and shCTRL SF188 cells, described in FIG. 3B, were allowed to plate in the presence of glutamine and then cultured in the absence of glutamine. Cell viability was determined at the time points shown by trypan blue dye exclusion. The data points shown represent the mean±SD of triplicate samples. (B) Myc-suppressed SF188 cells are resistant to an inhibitor of glutaminolysis. shMYC and shCTRL SF188 cells, described in FIG. 3B, were allowed to plate in the presence of glutamine and then were treated with 500 μM aminooxyacetate (AOA). Cell viability was determined at the time points shown by trypan blue dye exclusion. AOA-treated shCTRL cells were also treated with 7 mM dimethyl α-ketoglutarate (AOA+α-ketoglutarate). The data points shown represent the mean±SD of triplicate samples.

FIG. 7 depicts how glutaminolysis leads to the generation of NADPH. The two high affinity glutamine transporters (ASCT2, SN2) import glutamine into the cell. Once glutamine enters the cell, it can be metabolized through glutaminolysis. As used herein, glutaminolysis refers to the catabolic degradation of glutamine mediated by: deamidation of glutamine to glutamate via glutaminase (GLS) or the transamidation of glutamine to glutamate through the enzymes of nucleotide biosynthesis, the transamination of glutamate to α-ketoglutarate via transaminases such as alanine aminotransferase (ALT), the mitochondrial metabolism of α-ketoglutarate culminating in the production of malate, the oxidation of malate to pyruvate via malic enzyme (ME), and the reduction of pyruvate to lactate via lactate dehydrogenase A (LDH-A), leading to secretion of lactate into the extracellular medium. The steps of glutamine to glutamate to α-ketoglutarate may take place in the mitochondria. Whether NH4⁺ or NH₃ is the species that crosses the plasma membrane has not been determined. Importantly, glutaminolysis is a biosynthetically wasteful process in that the ammonia and lactate derived from glutamine are secreted from the cell. While not providing a significant source of biosynthetic precursors, glutaminolysis drives NADPH production, which fuels nucleotide and fatty acid biosynthesis. We propose that Myc activates glutaminolysis through transcriptional regulation of glutaminolytic enzymes.

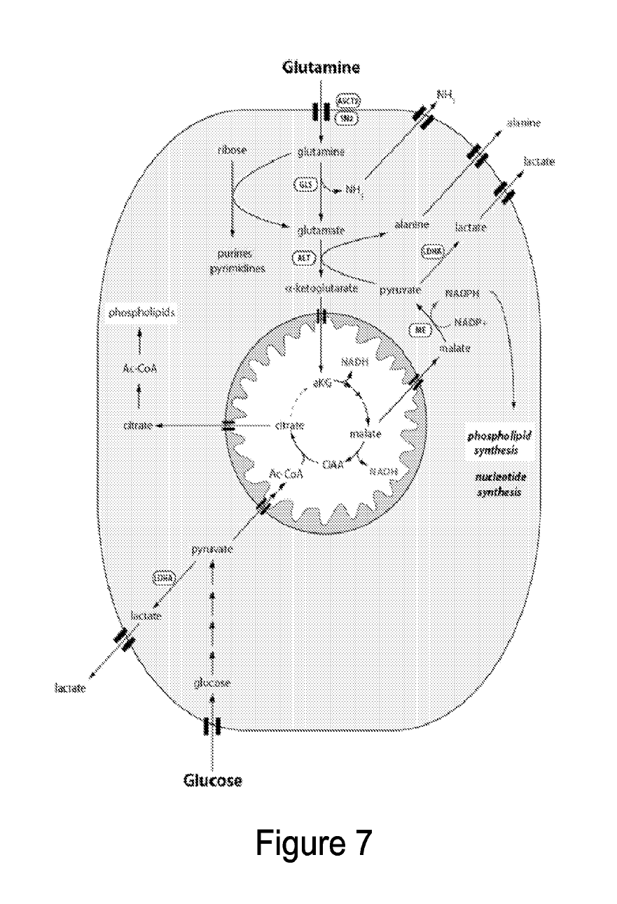

FIG. 8 presents pathways of glutamine metabolism in proliferating glioblastoma cells. The pathway begins with glutamine (Gln) in the upper right of the mitochondrion, and shows the contribution of glutamine carbon 3 (blue) into other metabolites in the mitochondrion and cytosol. These pathways supply the tricarboxylic acid (TCA) cycle and support the synthesis of fatty acids, proteins and nucleotides. Abbreviations: Ac-CoA, acetyl-CoA; Cit, citrate; α-KG, α-ketoglutarate; succ; succinate; Mal, malate; OAA, oxaloacetate; Asp, aspartate; Glu, glutamate; Pyr, pyruvate; Lac, lactate.

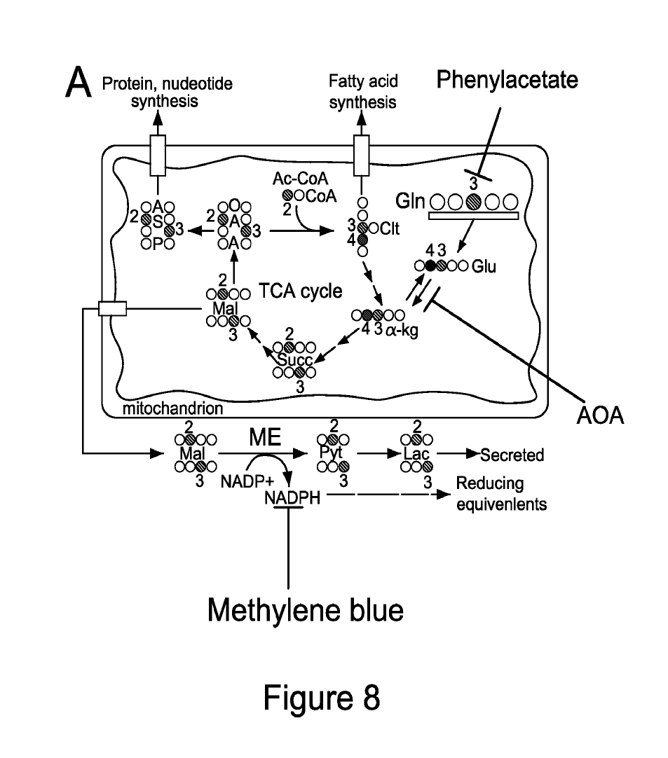

### DETAILED DESCRIPTION OF ILLUSTRATIVE EMBODIMENTS

In some aspects, the invention concerns methods of treating cancer comprising contacting the cancer cells with a chemical inhibitor of glutaminolysis. The present invention is based, in part, on the determination that glutaminolysis is an essential pathway for the growth and survival of Myc-overexpressing cancer cells. The research described herein details an array of pharmacological agents that impair glutaminolysis for the treatment of Myc-induced tumors. The data provided as an example demonstrate the specific cytotoxic effects of amino-oxyacetate (AOA) for Myc-transformed cells.

It was also found that glutaminolysis poses a toxic challenge to Myc-expressing cells due to the release of ammonium. Ammonium-mediated killing of Myc-expressing cells serves as a mechanism for the prevention of Myc-induced tumors ultimately circumvented through additional mutations. These additional mutations, such as those activating the PI3K/Akt pathway, can neutralize ammonium through the production of pyruvate. This pyruvate can neutralize ammonium through the action of alanine aminotransferase, an enzyme inhibited by AOA. As such, AOA treatment kills Myc-overexpressing cells, prior to the acquisition of further transforming mutations, making AOA a promising compound for the prevention of Myc-overexpressing tumors.

Pharmacological inhibition of glutaminolysis will be used to treat a variety of human cancers known to involve activation of the Myc oncogene, including but not limited to lung, breast, colon, prostate and lymphoma. Pharmacological inhibition of glutaminolysis may also be particularly useful for tumors that are FDG-PET negative, indicating low levels of glucose metabolism and suggesting the preferential use of glutamine metabolism. Such tumors include lymphomas, bronchoalveolar cell lung carcinoma, and carcinoid tumors. In addition, inhibition of glutaminolysis may be useful for treatment of pathological inflammatory states characterized by localized cell proliferation, such as auto-immune disease, hemophagocytic lymphohistiocytosis and infections. In addition, the agents described herein, such as AOA, will be used as an agent for the prevention of cancer.

We anticipate that the use of these compounds in clinical practice will follow an expedited path as these agents have already been FDA approved for other clinical indications.

Glutaminolysis is a critical pathway for a host of cancers, especially those with Myc overexpression. No current treatment modalities seek to specifically interrupt glutaminolysis. The compounds described herein have already been approved for other clinical uses at doses similar to those necessary for their anti-cancer properties with minimal side effects. As such, our technology represents a potent strategy for the prevention and treatment of cancer with limited side effects through the specific inhibition of glutaminolysis.

In the studies presented herein, the oncogenes known to contribute to malignant transformation of glial cells were tested for the ability to induce glutaminolysis. Here we report that the glutaminolytic phenotype exhibited by tumor cells correlates with a cellular addiction to glutamine metabolism for the maintenance of cell viability. In contrast to glucose, glutamine uptake was not found to be under the direct or indirect control of the PI3K/AKT pathway. Inhibitors of either PI3K or AKT, despite suppressing glucose metabolism in a dose-dependent fashion, had no effect on the glutaminolytic phenotype. In contrast, high level expression of Myc was required to maintain the glutaminolytic phenotype and addiction to glutamine as a bioenergetic substrate. When an inducible Myc transgene was introduced in mouse embryonic fibroblasts (MEF), induction of Myc expression resulted in the induction of glutamine transporters, glutaminase, and lactate dehydrogenase A (LDH-A). Induction of these key regulatory genes involved in glutaminolysis correlated with the Myc-induced increases in glutamine uptake and glutaminase flux. This increase in glutamine uptake was not a compensatory response to increased glutamine incorporation into proteins as a result of Myc-induced protein synthesis, as most of the additional glutamine carbon taken up following Myc induction was secreted as lactate. Myc-induced reprogramming of intermediate metabolism resulted in glutamine addiction, despite the abundant availability of glucose. Glutamine addiction correlated with Myc-induced redirection of glucose carbon away from mitochondria as a result of LDH-A activation. As a result, Myc-transformed cells became dependent on glutamine anapleurosis for the maintenance of mitochondrial integrity and TCA cycle function. Introduction of a Myc-shRNA hairpin reversed the glutamine dependence of Myc-transformed cells. In addition, Myc-transformed cells were sensitive to inhibitors of glutamate conversion to a-ketoglutarate in a Myc-dependent fashion and this sensitivity could be reversed by supplying cells with a cell-penetrant form of the mitochondrial substrate α-ketoglutarate. Taken together, these results suggest that glutamine addiction can be a direct consequence of Myc-induced transformation.

The invention is illustrated by the following examples that are not intended to be limiting.

### Methods

### Cell Culture and Media

SF188 cells (UC Brain Tumor Research Center, SF, CA) and SV40-immortalized MEF stably transfected with MycER (a gift from Drs. A T Tikhonenko and R. A. Amaravadi of University of Pennsylvania, Philadelphia, Pa.) were cultured in DMEM (Invitrogen), 10% FBS (Gemini Biosystems), 100 units/ml Penicillin, 100 ug/ml Streptomycin, 25 mM glucose, and 6 mM L-glutamine at 37° C. in a 5% CO₂ incubator. To avoid depleting oxygen, all experiments were carried out under subconfluent conditions. For glutamine starvation experiments, DMEM without glutamine (Invitrogen) was supplemented with 10% dialyzed FBS (Gemini Biosystems). For metabolic tracing experiments, DMEM without glutamine and with 10% dialyzed FBS was supplemented with either L-glutamine that was unenriched or with L-[U-¹³C₅]glutamine (Isotec), [U-¹⁴C₅]glutamine (GE Amersham), and [γ-¹⁵N]glutamine (Cambridge Isotope Laboratories). DMEM without glucose (Sigma) was supplemented with [U-¹⁴C₆]glucose (Sigma). To activate MycER, cells were incubated with 200 nM 4-hydroxytamoxifen for 24 hours.

### Glutamine Uptake

Cells were incubated with 4 mM glutamine and 1 μM [U-¹⁴C₅]glutamine in 2 ml of medium in a 6 well plate for 1 minute at 37° C. in a 5% CO₂ incubator. After incubation, the medium was aspirated and cells were washed three times with unlabeled medium, after which cells were lysed with 200 μL of a 0.2%SDS/0.2N NaOH solution, incubated for 1 hour, neutralized with 10 μL of 2N HCl, and analyzed with a beta scintillation counter (PerkinElmer Life Sciences). Bode B P & Souba W W (1994) Annals Of Surgery 220(4):411-424.

NMR analysis

Cells were grown to 80% confluency, after which the culture medium was removed and replaced with medium that contained 4 mM [U-¹³C₅]glutamine (and no unenriched glutamine) for 6 h. The resulting medium was then analyzed in a 20-mm NMR tube with a 9.4 Tesla spectrometer at 100.66 MHz. A 90° excitation pulse was applied every 20 seconds (fully relaxed), with broad-band decoupling used only during ¹³C data acquisition (no NOE enhancement). Spectra were acquired with 16384 points, a bandwidth of 25000 Hz and 3000 excitations. Free induction decays were apodized with exponential multiplication (3 Hz line broadening). Peak intensities in Fourier transformed spectra were determined with Nuts NMR (Acorn NMR, Livermore, Calif.). Carbon-3 of lactate derived from glutamine produced a doublet (21.5 and 20.3 ppm) due to splitting from ¹³C at carbon-2, while lactate derived from natural abundance ¹³C of glucose, produced a singlet at 20.9 ppm. All peaks were well resolved from each other.

### Lipid Biochemistry

To determine the rate of lipid synthesis from glucose or glutamine, 1-4×10⁵ MEF were plated in 6 well plates and cultured with DMEM supplemented with either 2.2 uM D-[U-¹⁴C₆] glucose (GE Amersham) or 0.54 uM L-[U-¹⁴C₅]glutamine (GE Amersham), both supplemented to 0.01% of their respective unenriched nutrients. After 8 h, cells were trypsinized, washed in PBS, and lysed in 0.4 ml of 0.5% Triton X-100. Total lipids were extracted and phospholipids were collected using thin-layer chromatography. DeBerardinis R J, et al. (2006) J. Biol. Chem. 281(49):37372-37380. Total incorporated label in the phospholipid fraction was analyzed with a scintillation counter (PerkinElmer Life Sciences).

qPCR

Total RNA was isolated using TRIzol Reagent (Invitrogen). 1 ug of RNA was used to prepare cDNA using SuperScript II Reverse Transcriptase (Invitrogen) according to the manufacturer's instructions. All samples were normalized to β-actin transcript levels. Murine glutaminase 1 (GLS1), glutamate dehydrogenase 1 (GLUD1), slc1a5 (ASCT2), and lactate dehydrogenase A (LDH-A) and Human slc1a5 (ASCT2), s1c38a5 (SN2), c-myc (MYC), eukaryotic translation initiation factor 1A (EIF1A), cyclin D2 (CYCLIN D2), and PITPNB phosphatidylinositol transfer protein, beta (BC018704) probes were synthesized by Integrated DNA Technologies. Samples were run on a 7300 Sequence Detection System (SDS) (Applied Biosystems) and analyzed using SDS 2.1 software.

### ChIP Analysis

SF188 cells were plated on 15-cm dishes and were fixed in 1% formaldehyde. Chromatin was sheared to an average size of 500-1,000 by by sonication (30 times with 30-s pulses, on a Diagenode Bioruptor). Lysates corresponding to 5-10×10⁶ cells were rotated at 4° C. overnight with 2 μg of polyclonal antibodies specific for c-MYC (sc-764, Santa Cruz Biotechnology) or normal rabbit IgG. Precipitated DNA fragments were quantified by using PCR.

### Immunoblotting

Cells were lysed in RIPA buffer (50 mM Tris-HCl at pH 7.4, 150 mM NaCl, 1% sodium deoxycholate, 0.1% SDS, 1× complete EDTA-free protease inhibitor cocktail tablets [Roche Applied Science]) and cleared lysates were separated on 4%-12% Bis-Tris gels (Invitrogen) followed by transfer onto nitrocellulose. After blocking in 3% skim milk, blots were probed using anti-c-Myc-N262 (1:1000; Santa Cruz Biotech) and anti-β-actin (1:10,000).

Akt inhibitor

SF188 cells stably expressing Bcl-x_{L} were plated at a density of 4×10⁵ cells in E-well plate format, and incubated in DMEM with 10% FCS for 36 hours prior to treatment with AKT inhibitor VIII (Calbiochem) at 0, 0.1, 2, 5, and 10 μM concentrations in triplicate. After 8 hours, medium samples were collected for metabolite analysis, and viable cells were counted using trypan blue exclusion.

### Lentiviral Transduction

Lentivirus was packaged in 293T cells co-transfected with pLKO.1-puro c-Myc or Luciferase-control shRNA plasmid (Sigma) in addition to the pVSV-G and pCMVdelta8.2 helper plasmids using lipofectamine 2000 (Sigma). Virus was collected from the culture medium filtered by Millex-HV(PVDF 0.45 μM) Syringe Driven Filter Unit (Millipore). Virus was then either frozen at −80° C. or directly added to target cells. Patel J H & McMahon S B (2007) J. Biol. Chem. 282(1):5-13.

### Glutamine's Contribution to Protein Synthesis

SF188 cells were cultured in medium supplemented with 0.01% [U-¹⁴C₅]glutamine relative to unenriched glutamine for 4 hours. Cells were washed three times with PBS, extracted using 0.5% Triton-X, acidified using 10% (v/v) of 35% (wt/v) sulfosalicylic acid, and then spun down at 13,000 RPM for 15 minutes at 4° C. The pellet was then resolubilized in NaOH at 37° C. ¹⁴C incorporated into the protein product were quantified using a scintillation counter (PerkinElmer Life Sciences). Inefficiency of protein recovery was controlled for by calculating the recovery of ¹⁴C bovine serum albumin (Sigma) added after lysis. Recovery of intracellular [U-¹⁴C₅]glutamine in its free form was controlled for by calculating the recovery of [u_{—}¹⁴C₅]glutamine added just prior to SSA precipitation. The recovered counts were sensitive to 5 ug/ml cycloheximide. The glutamine consumption rate was then calculated using the GLN2 glutamine assay kit (Sigma). The data presented are the mean +/−SD from four independent experiments.

### Media Metabolite Analysis

Media glucose, glutamine, lactate, and ammonia concentrations were analyzed using the Nova Biomedical Flex Analyzer (Waltham, Mass.).

### Gas Chromatography—Mass Spectrometry.

Cells were plated at 1.2×10⁶ per 6-cm dish. At 80% confluency, they were fed with 1.5 ml of DMEM containing 4 mM 1-[α-¹⁵N]glutamine (Cambridge Isotope Laboratories). Every 2 hours, medium was collected to determine the concentration of NH₃ using the Nova Biomedical Flex Analyzer (Waltham, Mass.). An aliquot was used to determine isotopic enrichment in NH₃ with published methods. Brosnan J T, et al. (2001) J. Biol. Chem. 276(34):31876-31882.

### AOA Inhibitor Experiments

SF188 cells were treated with AOA (Sigma) at doses ranging from 500 nM-500 μM and viability was assessed 24 h post-treatment. Moreadith R W & Lehninger A L (1984) J. Biol. Chem. 259(10):6215-6221. 500 μM was the lowest dose that killed a significant fraction of the cells. SF188 cells with Myc or control shRNA were replated 3 days post viral transduction. After allowing to plate overnight, cells were treated with 500 μM for 24 h, and then viability was assessed using trypan blue dye exclusion.

### Results

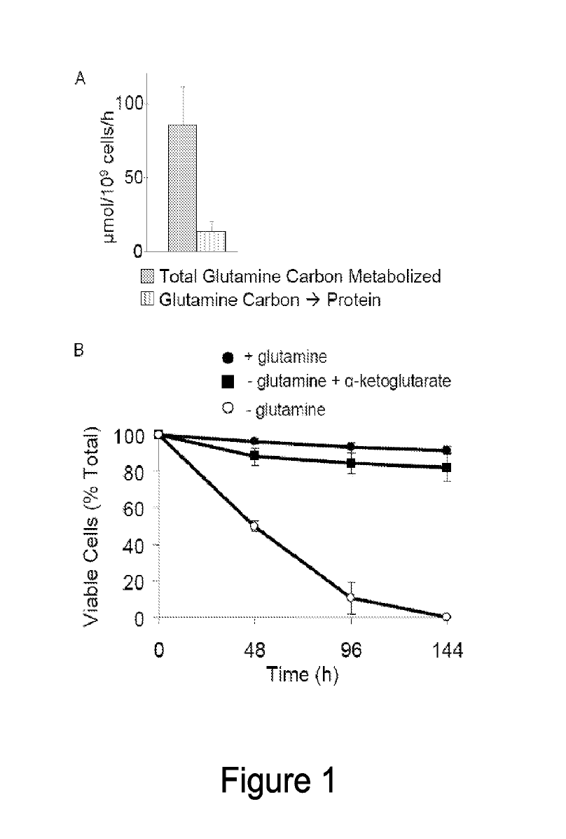

The human glioma line SF188 depends on glutamine catabolism to maintain viability. When SF188 cells were cultured in the presence of ¹⁴C-labeled glutamine, less than 15% of the glutamine the cells took up from the medium was incorporated into newly synthesized protein (FIG. 1A). Despite the fact that only a small fraction of the glutamine was used for anabolic synthesis, SF188 glioma cells were unable to survive in glutamine-deficient medium despite the presence of 25 mM glucose in the medium (FIG. 1B). α-ketoglutarate is the glutamine metabolite that enters the mitochondrial TCA cycle. The replacement of glutamine with a cell-penetrant form of α-ketoglutarate (dimethyl α-ketoglutarate) in the medium suppressed the cell death observed when the cells were cultured in glutamine-deficient medium (FIG. 1B). These data suggest that glutamine addiction does not result from glutamine's role as an amide donor in nucleotide biosynthesis or as the source of nitrogen for the maintenance of non-essential amino acid production since dimethyl α-ketoglutarate is devoid of nitrogen groups and cannot participate in these glutamine-dependent reactions.

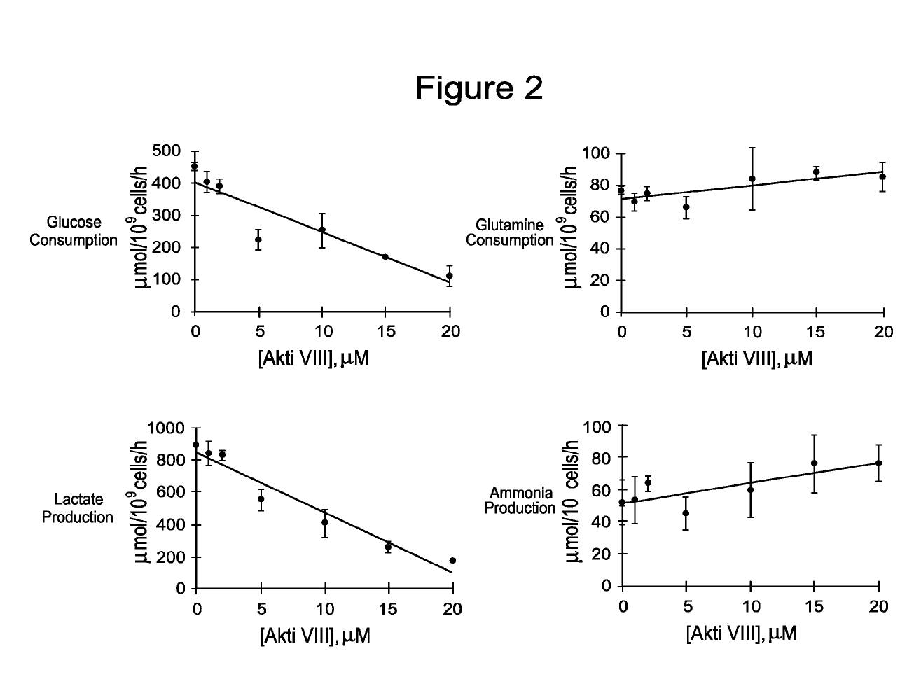

PI3K/AKT signaling regulates the consumption of glucose but not of glutamine in glioma cells. Previous work has demonstrated that the PI3K/AKT pathway can regulate the expression and surface translocation of a variety of nutrient transporters. Elstrom R L, et al. (2004) Cancer Res 64(11):3892-3899 and Edinger A L & Thompson C B (2002) Mol Biol Cell 13(7):2276-2288. We therefore investigated whether the PI3K/AKT pathway might also function to upregulate glutamine uptake and metabolism. To determine if PI3K/AKT signaling contributes to glutamine metabolism, the effects of the PI3K inhibitor LY294002 or the Akt inhibitor, Akt inhibitor VIII, on glucose and glutamine uptake were studied. SF188 cells with a Bcl-x_{L} transgene were used in this study to prevent apoptosis induced by drug treatment. As presented in FIG. 2, AKT inhibitor VIII suppressed glucose metabolism and lactate production in a dose-dependent fashion. In contrast, neither glutamine metabolism nor ammonia production was inhibited by AKT inhibitor VIII. If anything, there was a compensatory upregulation of glutamine metabolism in response to increasing doses of the inhibitor. Similar results were observed using the PI3K inhibitor LY294002.

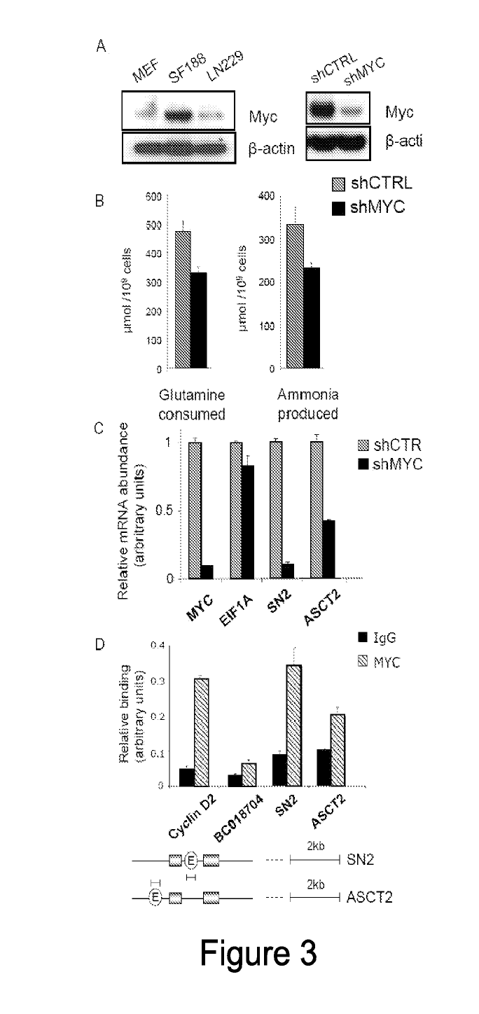

Myc can regulate glutaminolysis. Another oncogene associated with a poor prognosis in glial tumors is Myc. Ben-Porath I, et al. (2008) Nat Genet 40(5):499. SF188 cells were originally isolated from a patient whose tumor displayed amplification of Myc. Trent J, et al. (1986) PNAS 83(2):470-473. Western blot analysis of the SF188 cells utilized in these studies revealed Myc protein expression in excess of that observed in proliferating fibroblasts or a tumor cell line lacking amplified Myc (FIG. 3A). To determine if Myc is required for the maintenance of oxidative glutamine metabolism in SF188 cells, the effect of suppressing Myc using shRNA was investigated. SF188 cells were transduced with lentivirus containing shRNA against MYC (shMYC) or a lentivirus containing a control shRNA (shCTRL) and the rate of glutamine consumption and ammonia production was examined. shMYC cells had an approximately 80% reduction in their Myc level (FIG. 3A). This level of Myc reduction lead to a statistically significant reduction in glutamine consumption (P<0.01) and ammonia production (P<0.05) (FIG. 3B).

Myc activates the transcription of genes required for glutamine uptake and metabolism. Myc's transforming properties depend on its ability to bind to DNA and modify gene transcription. Dang CV (1999) Mol Cell Biol 19(1):1-11. Using quantitative RT-PCR (qPCR), we observed that shMYC cells expressed significantly lower levels of the high affinity glutamine importers ASCT2 and SN2 (P<0.01) without expressing significantly lower levels of the control transcript EIF1A (FIG. 3C). Furthermore, when Myc antibodies were used to perform chromatin immuno-precipitation (ChIP), Myc was found to selectively bind to the promoter regions of both ASCT2 and SN2 (FIG. 3D). This selectivity was comparable to that of the established Myc target CYCLIN D2 (FIG. 3D). Thus, Myc appears to bind to the promoter elements of glutamine transporters and this binding is associated with enhanced levels of glutamine transporter mRNA.

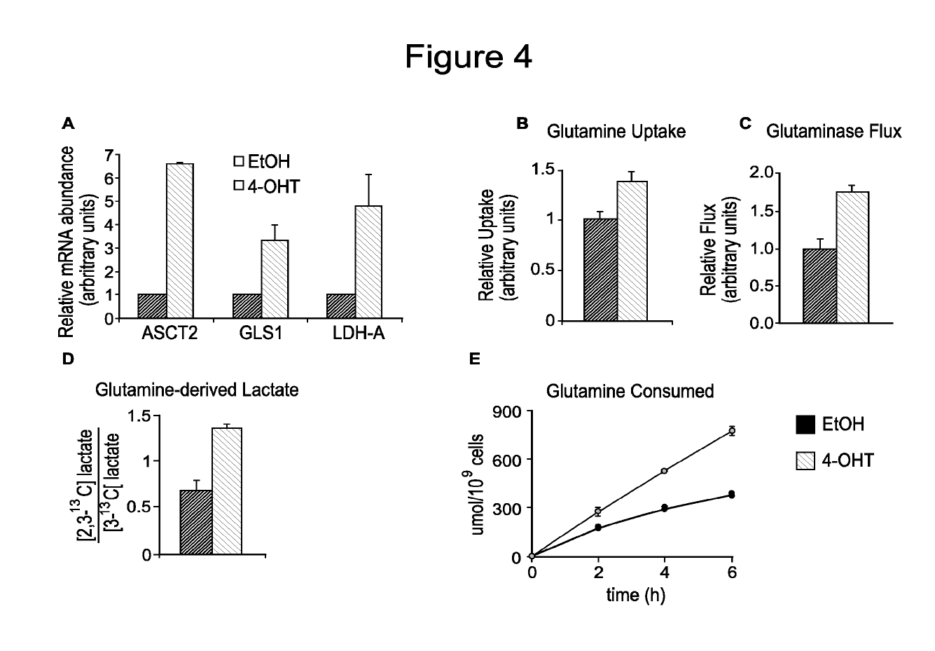

Myc activates glutaminolysis in MEF. The above data demonstrate that Myc transcription contributes to the high level of glutaminolysis exhibited by SF188 glioma cells. We next wanted to determine whether the induction of Myc transcription was sufficient to induce increased glutamine metabolism. To address this question, immortalized MEF that stably express a 4-hydroxy tamoxifen-inducible MycER construct (Thomas-Tikhonenko A, et al. (2004) Cancer Res 64(9):3126-3136) were analyzed to study the effects of Myc activation on glutamine metabolism. Treatment of cells with 4-hydroxy tamoxifen for 24 hours resulted in increased levels of the transcripts for not only the glutamine transporter ASCT2, but also for glutaminase (P<0.005), the enzyme that deamidates glutamine to glutamate, resulting in its intracellular capture, and LDH-A (P<0.005), which converts glutamine-derived pyruvate into lactate (FIG. 4A). These increases in mRNA levels correlated with enhanced functional activity. Treatment with 4-hydroxy tamoxifen resulted in statistically significant increases in glutamine uptake (P<0.05) (FIG. 4B), glutaminase flux (P<0.005) (FIG. 4C), and production of glutamine-derived lactate (P<0.05) (FIG. 4D) in the MEF expressing MycER. As a result, the rate at which glutamine was consumed from the medium was significantly greater (FIG. 4E). Furthermore, the glutamine uptake in vehicle-treated cells began to reach an asymptote by 6 hours, while the glutamine metabolized by the 4-hydroxy tamoxifen treated cells increased linearly over the culture period (FIG. 4E). Thus, the ability of Myc-induced cells to metabolize glutamine does not appear to be saturatable over this period as would be predicted for glutaminolysis, which ends not in the cellular accumulation of glutamine-derived metabolites over time, but in the secretion of glutamine-derived lactate into the medium. Despite increasing glutamine consumption, Myc induction did not increase the proliferative expansion of normal MEF under the same conditions. After 24 hours of 4-hydroxy tamoxifen or vehicle treatment of cells that were initially plated at 1×10⁵ cells/well the day prior to induction, the control cells increased to 5.2±0.5×10⁵ cells/well (mean±SD) while the Myc-induced cells had only increased to 3.5±0.5×10⁵ cells/well (mean±SD).

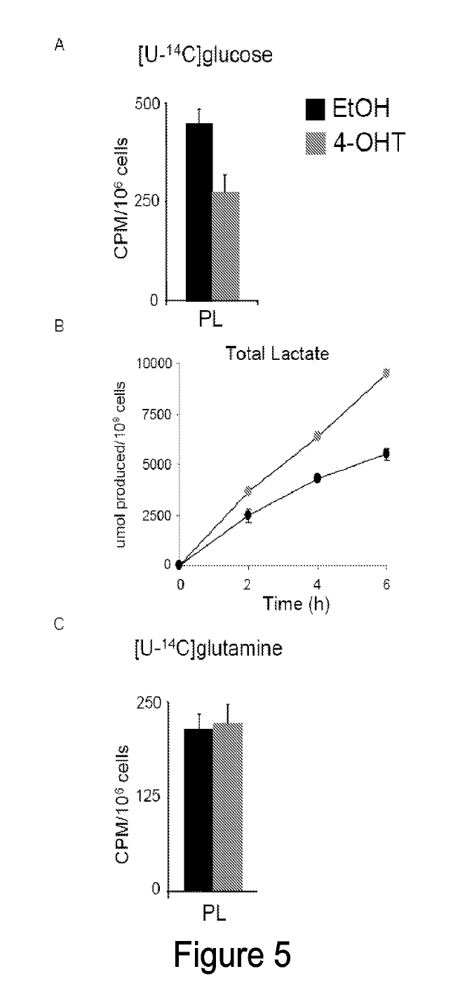

Myc diverts glucose away from mitochondrial metabolism. Previous studies have suggested that proliferating nontransformed cells maintain de novo phospholipid biosynthesis from glucose. See, Bauer D E, et al. (2005) Oncogene 24(41):6314. However, when MEF stably expressing MycER were treated with 4-hydroxy tamoxifen, the use of glucose as a precursor for phospholipid synthesis was suppressed (FIG. 5A). Concomitantly, an increased amount of the glucose-derived carbon was secreted from the cell as lactate (FIG. 5B). In contrast, the contribution of glutamine to phospholipid synthesis in Myc-induced cells was maintained and even increased despite increased secretion of glutamine-derived lactate (FIG. 5C; FIG. 4D). Together, these data demonstrate that Myc-induction leads to the diversion of glucose-derived pyruvate away from mitochondria, its conversion to lactate, and secretion from the cell. As a result, Myc induction enhances the cell's dependence on glutamine to maintain phospholipid synthesis and TCA cycle anapleurosis.

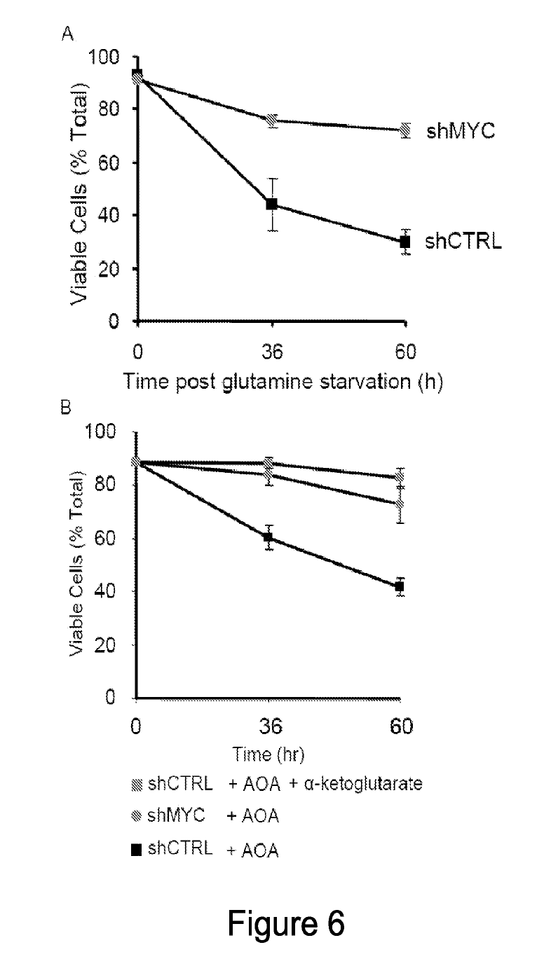

The glutamine addiction exhibited by SF188 glioma cells is Myc-dependent. The above data suggest that Myc is both necessary and potentially sufficient for the glutaminolytic metabolism exhibited by SF188 cells. To confirm that Myc is also involved in the glutamine addiction observed by these cells, SF188 cells were transduced with either a lentivirus containing a MYC-shRNA (shMYC) or a control shRNA (shCTRL). The resulting cells were incubated in glutamine-depleted or complete medium. Cells transduced with MYC-shRNA had a statistically significant increase (P<0.001) in their resistance to glutamine starvation relative to cells transduced with a control shRNA (FIG. 6A).

As a further confirmation that this glutamine addiction is Myc-dependent, the cells were treated with aminooxyacetate (AOA), a chemical inhibitor of glutamate-dependent transaminases that convert glutamate into α-ketoglutarate in the glutaminolytic pathway. Moreadith RW & Lehninger A L (1984) J. Biol. Chem. 259(10):6215-6221 AOA selectively induced the death of the cells transduced with control shRNA without affecting the viability of cells transduced with MYC-shRNA (P<0.01). Although AOA is a well-characterized inhibitor of the transaminases (Rej R (1977) Clin Chem 23(8):1508-1509), a chemical inhibitor can have non-specific effects on the viability of cells. To confirm the specificity of AOA's effects, the ability of dimethyl α-ketoglutarate to reverse the AOA-induced toxicity to SF188 cells was examined. Addition of 7 mM dimethyl α-ketoglutarate completely suppressed the death induced by AOA treatment of SF188 parental and control transduced cells (P<0.01) (FIG. 6B).

The factors that regulate glutamine uptake and metabolism during cell growth and transformation have remained poorly understood. In this manuscript, we provide evidence that oncogenic levels of Myc reprogram intermediate metabolism, leading to glutamine addiction for the maintenance of mitochondrial TCA cycle integrity. Previous work has demonstrated that LDH-A induction by Myc is required for Myc-transformation. Lewis B C, et al. (2000) Cancer Res 60(21):6178-6183. This results in diversion of glucose-derived pyruvate into lactate. Despite this, Myc-transformed cells display an increased mitochondrial mass and increased rate of O₂ consumption. Li F, et al. (2005) Mol Cell Biol 25(14):6225-6234. Furthermore, Morrish et al. (Morrish F, et al. (2008) Cell Cycle 7(8):1054-1066) have reported that Myc-overexpressing cells are exquisitely sensitive to inhibition of the mitochondrial electron transport chain. To explain this apparent paradox, they suggested that mitochondrial respiration might be maintained by catabolizing alternative bioenergetic substrates. Herein, we report that the alternative substrate is glutamine. Myc-transformation leads to conversion from glucose to glutamine as the oxidizable substrate utilized to maintain TCA cycle activity and cell viability. Myc binds to the promoters and induces the expression of several key regulatory genes involved in glutaminolytic metabolism. Our studies suggest that supraphysiological levels of Myc associated with oncogenic transformation are both necessary and sufficient for the induction of glutaminolysis to levels that result in glutamine addiction.

Yuneva et al. have previously reported that some, but not all, Myc transformants were dependent on glutamine. Yuneva M, et al. (2007) J. Cell Biol. 178(1):93-105. They also demonstrated that overexpression of Bcl-2 suppressed the death of Myc-transformants deprived of glutamine. Cell types and cell lines vary greatly in their level of expression in Bcl-2 family members and this may account for the differences observed between cell lines. Consistent with this, when SF188 cells were transfected with Bcl-x_{L}, they underwent cell cycle arrest but did not die when deprived of glutamine. Like Yuneva et al., we also found that cell-penetrant TCA cycle intermediates could suppress Myc-induced apoptosis. Together, these results suggest that glutamine addiction does not result from the use of glutamine as an amine donor, but rather because glutamine metabolism is essential to maintain mitochondrial integrity and function in Myc-transformed cells.

Whether levels of Myc expression induced in response to mitogenic stimulation also play a critical role in glutamine uptake and metabolism in the growth of nontransformed cells remains to be determined. At lower levels of expression, Myc-induced increases in protein synthesis and cell growth will also stimulate the incorporation of glutamine into newly synthesized proteins . Johnston L A, et al. (1999) Cell 98(6):779 and Iritani B M & Eisenman R N (1999) Proceedings of the National Academy of Sciences of the United States of America 96(23):13180-13185. There are undoubtedly additional signaling pathways that contribute to the regulation of glutamine uptake. Cells lacking oncogenic Myc levels, while not glutamine dependent, still take up sufficient glutamine to fuel both nucleotide and protein biosynthesis for cell growth and proliferation. Perhaps that is what is most surprising about the current results. Little of the glutamine uptake stimulated by Myc is utilized for macromolecular synthesis. Previous ¹³C-NMR studies found that during glutaminolysis, greater than 60% of glutamine-derived carbon is released from the cell as either lactate or CO₂. See, DeBerardinis R J, et al. (2007) Proceedings of the National Academy of Sciences 104(49):19345-19350. Although the TCA cycle was also replenished by glutamine, only 5% of glutamine fluxing through the TCA cycle was incorporated into fatty acids. Here we show that only 15% of the glutamine carbon taken up by the cell is incorporated in protein. Nevertheless, the additional stimulation of glutaminolysis by oncogenic levels of Myc results in cellular addiction to glutamine.

The ability of Myc to induce glutaminolysis does have a potentially beneficial effect for the transformed cell. Glutaminolysis results in the robust production of NADPH, thus providing an energy source for a wide variety of synthetic reactions required for cell growth. DeBerardinis R J, et al. (2007) Proceedings of the National Academy of Sciences 104(49):19345-19350. It has long been believed that the major source of NADPH production during cell growth occurs through the oxidative arm of the pentose phosphate shunt. However, recent evidence suggests that transformed cells exhibiting aerobic glycolysis derive the majority of their ribose biosynthesis through the non-oxidative arm of the pentose phosphate shunt. Serkova N & Boros L G (2005) Am J Pharmacogenomics 5(5):293-302. Under these conditions, G6PD cannot be used to produce a supply of NADPH to support macromolecular synthesis of fatty acids or nucleotides. Without a compensatory mechanism to generate NADPH, de novo nucleotide synthesis utilizing ribose produced in the non-oxidative arm of the pentose phosphate shunt would rapidly lead to intracellular depletion of NADPH. The ability of Myc to stimulate NADPH production through glutamine-dependent degradation provides the transformed cell with a mechanism to produce the quantities of NADPH needed to meet the demands of cell proliferation.

The data presented here also demonstrate that glutamine uptake is controlled independently of glucose uptake. Although the PI3K/AKT pathway plays a major role in regulating glucose uptake, it does not appear to be required for the uptake and catabolism of glutamine in Myc-transformed cells. Thus, the two main bioenergetic substrates used by proliferating cells appear to be under independent control by oncogenic signaling pathways. The remarkable ability of Myc and Aid to cooperate in transforming cells may result in part from their ability to complement each other in stimulating the uptake of these two critical nutrients.

The results presented here provide evidence that Myc transformation is associated with induction of a level of glutamine metabolism that results in glutamine addiction. Such addiction may ultimately be exploited through the use of inhibitors of the enzymes involved in the glutaminolytic pathway. The ability of the transaminase inhibitor AOA to induce the death of Myc-transformed cells but not isogenic cells in which Myc is suppressed by a MYC-shRNA provides the first evidence that such an intervention would have a selectively toxic effect on Myc-transformed cells. Myc-activation/amplification is one of the most common oncogenic events observed in a wide variety of cancers and is known to drive the progression of human lymphomas (Dalla-Favera R, et al. (1982) Proceedings of the National Academy of Sciences of the United States of America 79(24):7824-7827 and Taub R, et al. (1982) Proceedings of the National Academy of Sciences of the United States of America 79(24):7837-7841), neuroblastoma (Schwab M, et al. (1983) Nature 305(5931):245-248), and small cell lung cancer (Nau M M, et al. (1985) Nature 318(6041):69-73). Despite this, therapeutics that inhibit Myc's transcriptional properties have so far eluded drug discovery efforts. The identification of Myc's role in glutaminolysis may provide a number of enzymatic targets through which to selectively impair the growth and survival of Myc-transformed tumor cells. Finally, the present results demonstrate that nutrient uptake in mammalian cells is under distinct and specific regulation as a result of the properties of known oncogenes. Previous results have suggested that activating mutation in PI3K or AKT facilitate the uptake and metabolism of glucose in an mTOR-dependent manner. Edinger A L & Thompson C B (2002) Mol Biol Cell 13(7):2276-2288. In addition, induction of HIF-1 in response to hypoxia or mitochondrial ROS can also reprogram the intracellular fate of glucose. Lum J J, et al. (2007) Genes Dev 21(9):1037-1049. The present studies suggest that oncogenic Myc activation selectively induces addiction to glutamine, the other major catabolic substrate used by mammalian cells to maintain bioenergetics during cell growth and proliferation. Whether other essential nutrients are under similar control by these or other oncogenic signaling pathways remains to be determined.

What is claimed:

1. A method of treating cancer comprising contacting said tumors with a chemical inhibitor of glutaminolysis.

2. The method of claim 1, wherein said chemical inhibitor of glutaminolysis comprises one or more of amino-oxyacetate, phenylbutyrate, phenylacetate, and 3,7-bis(dimethylamino)-phenazathionium chloride (methylene blue).

3. The method of claim 1, wherein said cancer is cancer of the lung, breast, colon, or prostate.

4. The method of claim 1, wherein the cancer is lymphoma, bronchoalvedar cell lung carcinoma, or carcinold tumor.

5. A method of treating a Myc-induced tumor comprising contacting said tumors with a chemical inhibitor of glutaminolysis.

6. The method of claim 5, wherein said chemical inhibitor of glutaminolysis comprises one or more of amino-oxyacetate, phenylbutyrate, phenylacetate, and 3,7-bis(dimethylamino)-phenazathionium chloride (methylene blue).

7. The method of claim 5, wherein said tumor is associated with cancer of the lung, breast, colon, or prostate.

8. The method of claim 5, wherein the tumor is related to lymphoma, bronchoalvedar cell lung carcinoma, or carcinold tumor.

9. A method of inhibiting glutaminolysis in a Myc expressing cell comprising contacting said cell with one or more of amino-oxyacetate, phenylbutyrate, phenylacetate, and 3,7-bis(dimethylamino)-phenazathionium chloride (methylene blue).

10. A method of preventing cancer comprising contacting Myc expressing cells with one or more of amino-oxyacetate, phenylbutyrate, phenylacetate, and 3,7-bis(dimethylamino)-phenazathionium chloride (methylene blue).
